(19) United States
(12) Patent Application Publication
(10) Pub. No.: US 2006/0161192 A1
(43) Pub. Date: Jul. 20, 2006

(54) Wound needle with composite cutting edge

(76) Inventor: Christopher S. Young, South Kent, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 10/933,997

(22) Filed: Sep. 3, 2004

(51) Int. Cl. A61B 17/34

(52) U.S. Cl. 606/185

(57) ABSTRACT

A wound needle comprising an elongated shank having a proximate end and a distal end; a piercing member disposed at the distal end of the elongated shank, wherein the piercing member comprises an elongated channel into which the distal end is disposed; wherein a cutting section is formed by both the piercing member and the distal end of the elongated shank, wherein the distal end of the elongated shank comprises two converging faces which form a portion of the cutting section and wherein the piercing member comprises at least two converging faces which form a portion of the cutting section; wherein the elongated shank and the piercing member are comprised of different materials; wherein a portion of the cutting section formed by the at least two converging faces of the piercing member extends further outwardly from the distal end of the elongated shank than does the portion of the cutting section formed by the converging faces of the elongated shank; and wherein the respective converging faces of the elongated shank are at least essentially flush with the respective converging faces of the piercing member; whereby cutting is initiated by the portion of the cutting section formed by the piercing member and continued at least in part by the portion of the cutting section formed by the converging faces of the elongated shank.

### BACKGROUND OF THE INVENTION

The invention relates generally to wound needles, and in particular, to would needles which have a cutting edge comprised of both a portion of a piercing member, made from material such as stainless steel, and a portion of an elongated shank, made for example from plastic, to which the piercing member is coupled.

Wound needles are well known in the art. In the most typical form, they comprise an elongated shank made of stainless steel with a distal end having converging faces that terminate in a cutting edge. Disadvantageously, a cutting edge formed completely from stainless steel tends to be expensive to manufacture due to the difficulty in grinding the elongated cutting edge. The prior art also recognizes the idea of putting a stainless steel cutting tip on a plastic shank, but this too requires an undesirable amount of precision to form the cutting edge out of the steel. Making the entire wound needle from plastic is also undesirable since a sufficiently sharp tip cannot be formed from conventional plastic.

Accordingly, it remains important to maintain a sharp tip made of a sufficiently hard material, such as by way of example and not limitation, stainless steel. However, the present inventor has recognized that a full stainless steel cutting edge is not necessarily needed once the initial incision is made. Accordingly, providing a portion of the cutting edge from a material other than steel, such as by way of example, plastic, would overcome the aforementioned difficulty of providing a satisfactory cutting edge over the length thereof at a reasonable cost of manufacture.

Hence, it would be advantageous to provide a wound needle that has the necessary precision edge for the initial cut and equally provides the precision edge for the remainder of the cutting edge, all at a cost of manufacture and construction that meets and/or exceeds the rigid constraints and requirements of the medical industry. It is believed that the present invention provides the foregoing advancements in the art, as well as achieves the objectives set forth below.

### SUMMARY AND OBJECTIVES OF THE INVENTION

Accordingly, it is an objective of the present invention to provide an improved wound needle construction and method of making same.

Another objective of the present invention is to provide an improved wound needle that overcomes the deficiencies in the prior art.

For example, it is an object of the present invention to provide an improved wound needle that comprises a sufficiently sharp cutting edge and which is relatively inexpensive to manufacture.

Yet another objective of the present invention is to provide an improved wound needle that is inexpensive to manufacture and which further meets and/or exceeds the rigid constraints and requirements of the medical industry.

Still other objects and advantages of the invention will in part be obvious and will in part be apparent from the specification.

The invention accordingly comprises the features of construction, combination of elements and arrangement of parts and sequence of steps which will be exemplified in the construction, illustration and description hereinafter set forth, and the scope of the invention will be indicated in the claims.

### BRIEF DESCRIPTION OF THE DRAWINGS

For a fuller understanding of the invention, reference is had to the following description taken in connection with the accompanying figures, in which:

FIG. 1 is a perspective view of a wound needle constructed in accordance with the present invention;

FIG. 2 is an elevational view of a piercing member prior to the injection molding of the shank;

FIG. 3 is a top plan view of the piercing member of FIG. 2;

FIG. 4 is an elevational view of the needle of FIG. 1; and

FIG. 5 is a top plan view of the needle illustrated in FIG. 4.

Like features in the various figures will have like numbers but not every feature is called out with a reference numeral in each figure.

### DETAILED DESCRIPTION OF THE PREFERRED EMBODIMENTS

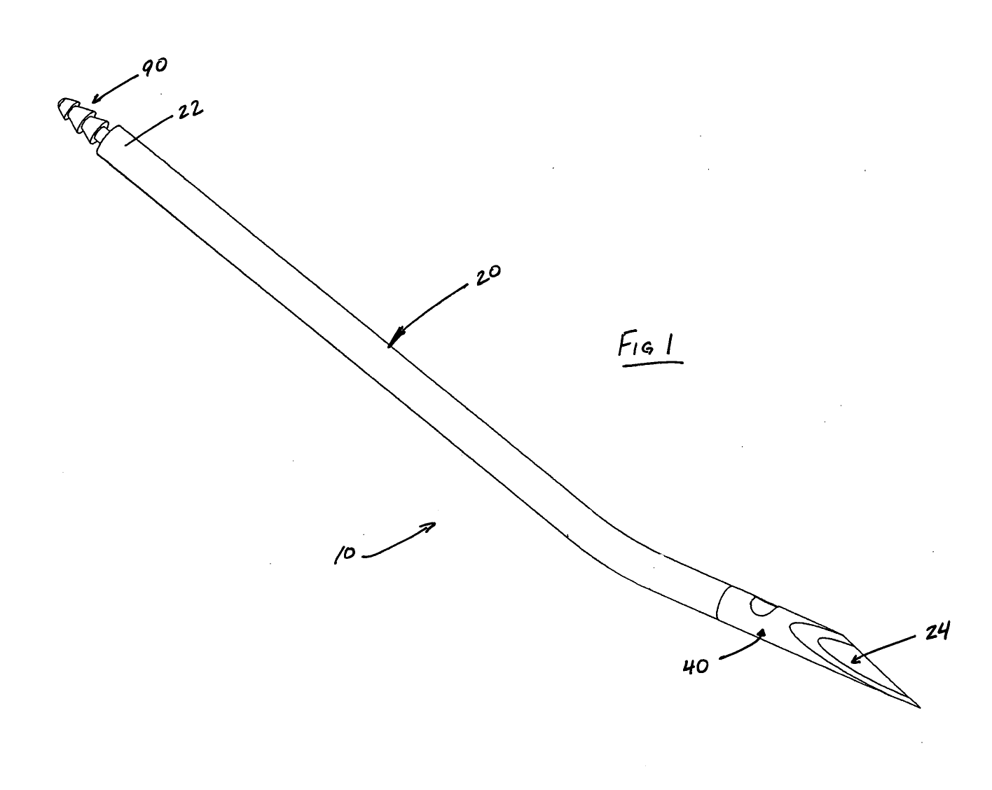

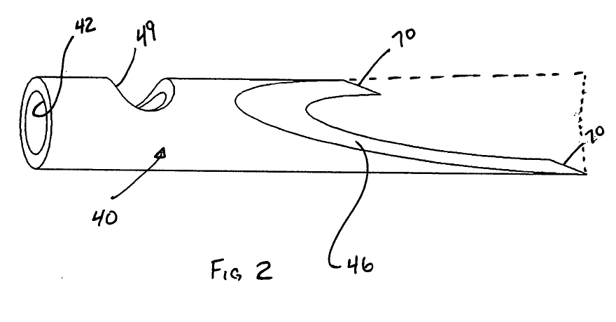

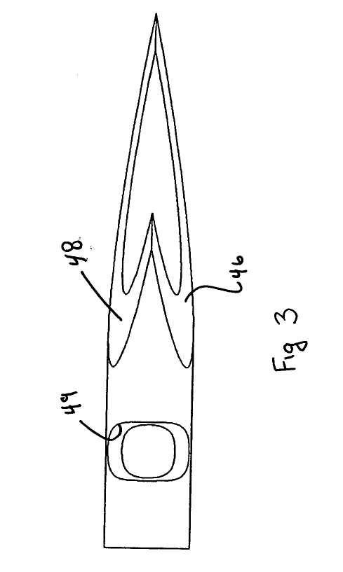

Reference is first made to FIG. 1, which illustrates a wound needle, generally indicated at 10, constructed in accordance with the present invention. As illustrated therein, wound needle 10 (also referred to as “needle 10”), comprises an elongated shank generally indicated at 20 and having a proximate end 22 and a distal end generally indicated at 24, and a piercing member, generally indicated at 40, disposed at distal end 24 of elongated shank 20. To secure elongated shank 20 to piercing member 40, piercing member 40 preferably comprises an elongated channel 42 (see FIGS. 2, 3) into which distal end 24 of elongated shank 20 is inserted during a molding process, to be disclosed further below.

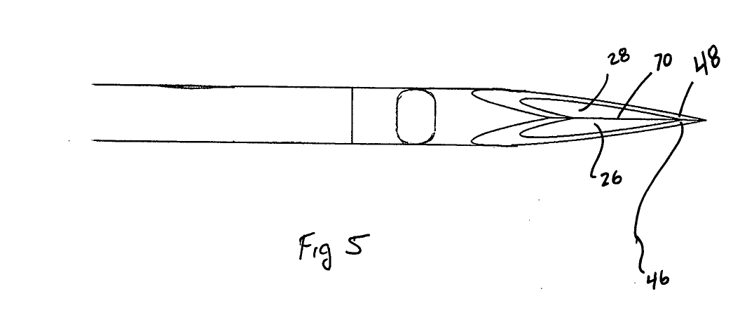

Needle 10 comprises a cutting section, generally indicated at 60, and defined by cutting edge 70 (FIGS. 2 and 5) which can be seen easily in FIGS. 1, 4 and 5 as being formed by both piercing member 40 and distal end 24 of elongated shank 20. It can be seen that distal end 24 of elongated shank 20 comprises two converging faces 26, 28 which form a portion of cutting section 60, namely that portion indicated by reference number 62, and wherein piercing member 40 comprises at least two converging faces 46, 48 which form a portion of cutting section 60, namely that portion indicated by reference number 64.

Importantly, the respective converging faces 26, 28 of elongated shank 20 are at least essentially flush with the respective converging faces 46, 48 of piercing member 40. That is, face 26 of elongated shank 20 is at least essentially if not as perfectly as possible, flush with face 46 of piercing member 40 and face 28 of elongated shank 20 is at least essentially if not as perfectly as possible, flush with face 48 of piercing member 40. This is important to provide as smooth a transition as possible from the material comprising piercing member 40 to the material comprising elongated shank 20 during the insertion into the skin of cutting section 60.

Also important to the present invention is the fact that the outwardly most portion 64 of the cutting section 60 (i.e. the portion of the cutting section that makes the initial incision) is comprised of the material comprising piercing member 40. That is it can be seen that in the preferred embodiment, portion 64 of cutting section 60 extends further outwardly from distal end 24 of elongated shank 20 than does portion 62 of cutting section 60. This is an important so that cutting is initiated by the portion (i.e. portion 64) of cutting section 60 formed by piercing member 40 and continued at least in part by the portion (i.e. portion 62) of cutting section 60 formed by converging faces 26, 28 of elongated shank 20.

It should be understood that converging faces 46, 48 preferably converge at two sections, namely portion 64 of cutting section 60 and at a portion designated by reference numeral 66, which is adjacent portion 62. In this way, continuous edge line 70 (FIGS. 2 and 5) is formed along portions 64, 62, 66.

With the finalized construction of needle 10 now fully disclosed, reference will now be made to the Figures in connection with the following disclosure for an understanding of the preferred method of forming needle 10.

Generally speaking, the preferred method of forming wound needle 10 from elongated shank 20 and piercing member 40 comprises several steps, the order of which is not material to the present invention nor are the claims limited thereby, although the following shall constitute a preferred preference of order.

For example, in one of the early steps of the preferred method, a piercing member 40, which at the outset, is generally tubular in shape, will undergo a grinding operation to form the above-mentioned respective converging face 46 and face 48. The preferred grinding operation required to form converging faces 46, 48 may be carried out on a conventional grinding machine of the type well known in the art. In a first grinding step, face 46 will be ground at a 10° grind angle. Then member 40 will be rotated on its axis 120° and ground again at a 10° grind angle.

Next, the finalized shape of piercing member 40 is placed in a mold where an injectable polymer (i.e. that which forms elongated shank 20) is injection molded therein, with a portion of the pre-hardened material flowing into channel 42. As one skilled in the art should appreciate, the mold could be shaped such that converging faces 26, 28 are formed during the molding process. Alternatively, converging faces 26, 28 could be formed during a latter cutting and/or grinding step, similar to that which forms faces 46, 48. In this way, portion 62 of cutting section 60 is formed. Since it is highly preferable to ensure as best as possible that the respective converging faces 26, 28 of elongated shank 20 are formed at least essentially, if not fully, flush with the respective converging faces 46, 48 of piercing member 40, it may be necessary to slightly grind, cut, smooth or otherwise even manually shape faces 26 and 28 to form the final cutting edge.

From this process, portion 64 formed by the converging faces 46, 48 of piercing member 40 extend further outwardly from distal end 24 of elongated shank 20 than does portion 62 of cutting section 60 formed by converging faces 26, 28 of elongated shank 20. In this way, cutting by the cutting edge (e.g. of skin) will be initiated by portion 64 by virtue of its further extension outwardly from distal end 24 of elongated shank 20 and continued at least in part by portion 62 of cutting section 60. As clearly illustrated, further continued insertion will cause the cutting of the skin by portion 66 of cutting section 60.

As should now be appreciated, a wound needle constructed and/or manufactured as set forth herein overcomes the deficiencies in the prior art. For example, a wound needle as constructed and/or formed as set forth herein will have a sufficiently sharp cutting edge and yet be relatively inexpensive to manufacture. In fact, such a wound needle will meet and/or exceed the rigid constraints and requirements of the medical industry.

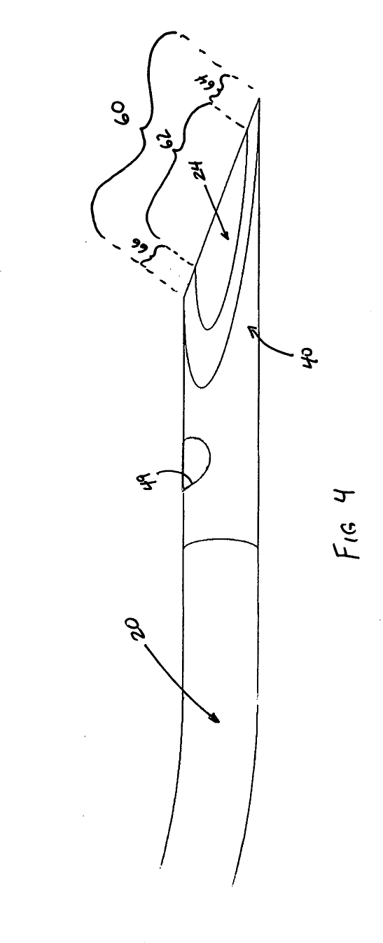

Still further features and advantages are provided by the invention disclosed herein. For example, a notch 49 (see FIGS. 2 and 4) may be provided in a portion of piercing member 40, which can be formed by another grinding or cutting operation. During the molding process, the flowing polymer material (i.e. the material that forms elongated shank 20) will flow into the void created by notch 49. In this way, piercing member 40 may be effectively restrained from separation from elongated shank 20 by the inability of piercing member 40 to slide over the material positioned in notch 49.

As set forth above, piercing member 40 is preferably made of stainless steel and elongated shank 20 is preferably comprised of plastic, although suitable alternatives may be substituted thereby as would be known by one skilled in the art. Lastly, to be sure, a series of integrally formed conical portions (FIG. 1) generally indicated at 90, may be provided for reliable securing of a tube thereon in order to carry out the procedure of wound drainage to which the present invention is directed.

Lastly, the use of a plastic shank provides for many advantages heretofor unavailable. For example, during the molding process, shank 20 can be fluted to add strength and/or rigidity thereto. Such fluting and/or shaping of the shank can also add to lower shank drag.

It will thus be seen that the objects set forth above, among those made apparent from the preceding description, are efficiently attained and, since certain changes may be made in the above constructions and methodologies without departing from the spirit and scope of the invention, it is intended that all matter contained in the above description or shown in the accompanying drawings shall be interpreted as illustrative and not in a limiting sense.

It is also to be understood that the following claims are intended to cover all of the generic and specific features of the invention described herein and all statements of the scope of the invention that as a matter of language might fall therebetween.

What is claimed is:

1. A wound needle comprising:
an elongated shank having a proximate end and a distal end;
a piercing member disposed at the distal end of the elongated shank, wherein the piercing member comprises an elongated channel into which the distal end is disposed;
wherein a cutting section is formed by both the piercing member and the distal end of the elongated shank, wherein the distal end of the elongated shank comprises two converging faces which form a portion of the cutting section and wherein the piercing member comprises at least two converging faces which form a portion of the cutting section;
wherein the elongated shank and the piercing member are comprised of different materials;
wherein a portion of the cutting section formed by the at least two converging faces of the piercing member extends further outwardly from the distal end of the elongated shank than does the portion of the cutting section formed by the converging faces of the elongated shank; and
wherein the respective converging faces of the elongated shank are at least essentially flush with the respective converging faces of the piercing member;
whereby cutting is initiated by the portion of the cutting section formed by the piercing member and continued at least in part by the portion of the cutting section formed by the converging faces of the elongated shank.

2. The wound needle as claimed in claim 1, wherein the piercing member comprises a notch into which material forming the elongated member will flow during a molding process;
wherein the piercing member is at least in part restrained from separation from the elongated shank by the inability of the piercing member to slide over the material positioned in the notch.

3. The wound needle as claimed in claim 1, wherein the elongated shank is comprised of plastic and the piercing member is comprised of stainless steel.

4. A method of forming a wound needle from an elongated shank having a proximate end and a distal end, and a piercing member having a channel into which the distal end is disposed, wherein the piercing member is positioned at the distal end of the elongated shank and wherein the elongated shank and the piercing member are comprised of different materials, the method comprises the steps of:
forming at least two converging faces at a distal end of the piercing member which comprises a portion of a cutting section;
molding a portion of the elongated shank into the channel of the piercing member and forming at least two converging faces which comprise another portion of the cutting section, wherein the respective converging faces of the elongated shank are formed at least essentially flush with the respective converging faces of the piercing member;
wherein a portion of the cutting section formed by the converging faces of the piercing member extend further outwardly from the distal end of the elongated shank than does the portion of the cutting section formed by the converging faces of the elongated shank;
whereby cutting by the cutting section will be initiated by the portion of the cutting section formed by the piercing member by virtue of its further extension outwardly from the distal end of the elongated shank and continued at least in part by the portion of the cutting section formed by the converging faces of the elongated shank.

5. The method as claimed in claim 4, including the step of forming the at least two converging faces of the distal end by a grinding or a cutting operation.

6. The method as claimed in claim 4 including the step of forming the at least two converging faces of the distal end during the molding step
